(19) United States
(12) Patent Application Publication
(10) Pub. No.: US 2007/0196519 A1
(43) Pub. Date: Aug. 23, 2007

(54) Method for making therapeutic compositions containing trimethoprim and cranberry extract for treating urinary tract infections

(76) Inventor: Robert B. Royds, Plainsboro, NJ (US)

Correspondence Address: Kenneth Watov, Esq.;WATOV & KIPNES, P.C., P.O. Box 247, Princeton Junction, NJ, 08550, US

(21) Appl. No.: 11/788,136

(22) Filed: Apr. 19, 2007

Related U.S. Application Data
(62) Division of application No. 11/333,179, filed on Jan. 17, 2006.

(51) Int. Cl. A61K 36/45; A61K 31/505

(52) U.S. Cl. 424/732; 514/275

(57) ABSTRACT

A method for making an over-the-counter therapeutic composition for treating urinary tract infections in warm-blooded animals, including humans, includes the steps of obtaining a combination of trimethoprim or pharmaceutically salts thereof and cranberry extract in an effective amount to prevent, reduce or eliminate symptoms associated with the infection, and a pharmaceutically acceptable carrier, and mixing at least one liter of water therewith.

### RELATED APPLICATION

This Application is a Divisional from co-pending prior application Ser. No. 11/333,179 filed on Jan. 17, 2006, the teachings of which are incorporated by reference herein to the extent they do not conflict herewith.

### FIELD OF THE INVENTION

The present invention relates to therapeutic compositions, and more particularly to a therapeutic composition containing trimethoprim and cranberry extract for the treatment of urinary tract infections.

### BACKGROUND OF THE INVENTION

Urinary tract infections (UTIs), or cystitis, pose a serious health problem affecting millions of people each year. The disorder generally affects the kidneys, the ureters, the bladder, and the urethra. More than 90% of acute UTIs in patients with normal anatomical structure and function are caused by certain strains of E. coli. Ten to twenty percent are caused by coagulase-negative Staphylococcus saprophyticus, and five percent or less are caused by other enterobacteriaceae organisms or enterococci. In rare cases, Candida albicans can cause UTI in diabetic patients. S. saprophyticus is the second most common cause in young sexually active women.

Most UTIs, specifically uncomplicated UTIs, can be treated with prescription oral antibiotics such as ampicillin, co-trimoxazole, trimethoprim, macrodantin, or cephalexin. More aggressive treatments include ciprofloxacin. Trimethoprim (2,4-diamino-5-(3,4,5-trimethoxybenzyl)pyrimidine) or its salt is a bacteriostatic antibiotic mainly used in the prophylaxis and treatment of urinary tract infections. It belongs to the class of chemotherapeutic agents known as dihydrofolate reductase inhibitors. Trimethoprim was formerly marketed by GlaxoWellcome under trade names including Proloprim®, Monotrim® and Triprim®; but these trade names have been licensed to various generic pharmaceutical manufacturers.

Trimethoprim is also used in combination with sulfamethoxazole, a sulfonamide antibiotic. This combination, known as co-trimoxazole, exhibits a synergistic antibacterial effect. Although the combination has been effective in antibiotic treatments, its use has been declining due to reports of sulfamethoxazole bone marrow toxicity. Co-trimoxazole, owing to its greater efficacy, is indicated for a wider range of infections. For example, it is used as prophylaxis in patients at risk for Pneumocystis jiroveci pneumonia (e.g. AIDS patients and those with some hematological malignancies), as therapy in Whipple's disease and certain other infections. The oral antibiotics generally require a prescription order from the attending physician for administration to the patient.

It has also been known that cranberry juice and derivatives thereof have exhibited benefits to health. Cranberry juice is a pleasant, palatable beverage generally enjoyed by a majority of the population. Recent evidence suggests that cranberry juice contains an ingredient exhibiting pharmacological activity that imparts therapeutic benefits in cases of urinary tract infections. Thus, doctors often recommend cranberry products to patients suffering from urinary tract infections. It is believed that cranberry juice inhibits the adhesion of bacteria to mammalian cells such as epithelial cells. This inhibition of bacterial attachment to the epithelial cells including the cells lining the urinary tract promotes the shedding of pathogenic bacteria, thus treating and preventing infections.

There is a need for devising safe and more effective methods and therapeutic compositions for treating and preventing urinary tract infections. There is a further need to provide methods and therapeutic compositions for treating and preventing urinary tract infections available in the form of “over-the-counter” remedies for permitting greater access and availability to consumers. Furthermore, there is a need to find more effective means for enhancing the efficacy of existing therapeutic agents such as trimethoprim against pathogenic bacteria, while imparting minimal undesired effects typically associated with therapeutic agents that typically require prescriptions. There is a need for implementing such treatment and prevention in a simple, reliable and cost effective manner for greater accessibility to those suffering from urinary tract infection.

### SUMMARY OF THE INVENTION

The present invention relates generally to a therapeutic composition and method for treating and preventing of urinary tract infections in warm-blooded animals, including humans. The present invention more specifically relates to the use of an extract of a plant belonging to the genus Vaccinium referred hereinafter as “cranberry extract” in combination with a therapeutic agent such as trimethoprim or a pharmaceutically acceptable salt thereof. This combination is used to prepare the therapeutic composition useful for treating and preventing urinary tract infections in warm-blooded animals, including humans. The resulting therapeutic composition can be provided, for example, as a beverage drink, or as a solid such as a powder drink mix for preparing a beverage drink through the addition of a liquid.

The therapeutic composition of the present invention generally comprises a combination of trimethoprim or a pharmaceutically acceptable salt thereof, and cranberry extract in amounts sufficient to treat urinary tract infections in warm-blooded animals, including humans. Trimethoprim belongs to a class of 5-benzyl-2,4-diaminopyrmimidines which exhibit valuable antibiotic properties useful in the treatment of bacterial and protozoan diseases. The present composition containing trimethoprim exhibits enhanced treatment efficacy and antimicrobial activity when complemented with cranberry extract. Accordingly, lower amounts of trimethoprim can be used with little or no appreciable reduction in antimicrobial effects to yield a safer product that can be formulated for “over the counter” dispensing and administration.

The active components of the cranberry extract are theorized to induce an anti-adhesion effect on pathogenic bacteria suspected of causing urinary tract infection, and the extract is generally isolated from plant material and juice from berries of plant species of the genus Vaccinium including, but not limited to, Vaccinium macrocarpon, Vaccinium microcarpum and Vaccinium oxycoccus. The cranberry extract can be in the form of cranberry concentrates, cranberry fruits, cranberry fruit concentrates, cranberry juice, cranberry juice concentrate, cranberry juice extract, cranberry juice powder, cranberry powder, cranberry powder extract, and the like.

In one aspect of the present invention, there is provided a therapeutic composition for treating or preventing urinary tract infections in warm-blooded animals, including humans, comprising a combination of trimethoprim or pharmaceutically acceptable salts thereof and cranberry extract, in an effective amount to prevent, reduce or eliminate symptoms associated with the infection, and a pharmaceutically acceptable carrier.

In another aspect of the present invention, there is provided a method for treating or preventing a urinary tract infections in warm-blooded animals, including humans, comprising administrating a therapeutic composition of the present invention to the warm-blooded animal in an effective amount for a time sufficient to prevent, reduce or eliminate symptoms associated with the infection.

### DETAILED DESCRIPTION OF THE INVENTION

The present invention is generally directed to a therapeutic composition and method for treating and preventing of urinary tract infections in warm-blooded animals, including humans. The present invention more specifically relates to the use of an extract of a plant belonging to the genus Vaccinium, referred hereinafter as “cranberry extract”, in combination, with a therapeutic agent such as trimethoprim or a pharmaceutically acceptable salt thereof, to prepare the therapeutic composition useful for treating and preventing of urinary tract infections in warm-blooded animals, including humans. The combination of components of the present invention will have complementary activity to enhance the effectiveness of the individual components in the treatment and prevention of urinary tract infections in warm-blooded animals, including humans.

The combination of cranberry extract and trimethoprim in the composition enhances the treatment efficacy against urinary tract infections with improved therapeutic activity as compared to trimethoprim administered alone. The composition of the present invention can be readily formulated for “over the counter” dispensing and administration. The term “over the counter” simply means the composition is determined in accordance with the provisions of the U.S. Food and Drug Administration to be used primarily to treat or prevent a condition that does not require the direct supervision of a physician and is proven to be reasonably safe and well-tolerated with little abuse potential.

Cranberries are a group of evergreen dwarf shrubs in the genus Vaccinium. There are known three species of cranberry: Vaccinium oxycoccus, Vaccinium microcarpum, and Vaccinium erythrocarpum. The cranberry extract is obtained from plant material such as the fruit, leaves and berries from the plant species of the genus Vaccinium, which is believed to contain active ingredients having activity to inhibit the adherence of certain bacterial species to various substrates. The extract can be in liquid form that can be administered to the patient, or in a solid form such as, for example, a powder or granule mixture or an effervescent tablet that can be subsequently dissolved in a suitable solvent for oral administration.

In one embodiment of the present invention, there is provided a therapeutic composition for treating or preventing urinary tract infections in warm-blooded animals, including humans, comprising a combination of trimethoprim or pharmaceutically acceptable salts thereof and cranberry extract, in an effective amount to prevent, reduce or eliminate symptoms associated with the infection, and a pharmaceutically acceptable carrier.

In a preferred embodiment, the therapeutic composition is in the form of a beverage for oral consumption by the warm-blooded animal. It is noted that solutions are preferred over solid forms for oral administration, since drugs in solution are more rapidly absorbed. Solutions are also often more acceptable to patients, in terms of palatability. Furthermore, increased fluid intake increases urinary output, the increased flow promoting pathogen elimination.

As used herein, the term “trimethoprim” encompasses the compound 2,4-diamino-5-(3,4,5-trimethoxybenzyl)pyrimidine, as well as its pharmaceutically acceptable salts, hydrates and complexes. (See “9782. Trimethoprim”, The Merck Index, 13^{th} Ed. (2001, pp. 1730). The term “pharmaceutically acceptable salts thereof” is intended to mean salts formed in connection with trimethoprim utilizing suitable mineral acids such as hydrochloric acid, sulfuric acid, and the like, and organic acids such as acetic acid. Trimethoprim “complexes” refer to chemical complexes of trimethoprim with other chemical constituents that result in entities that retain at least a substantial portion of the antimicrobial activity of trimethoprim. A method for preparing trimethoprim is disclosed in U.S. Pat. No. 2,909,522, to Hitchings et al., issued Oct. 20, 1959.

The term “cranberry extract” encompasses any wet or dried portion of the plant material of various plant species including members of the genus Vaccinium, preferably the portions that are rich in the active agents such as the leaves or fruit in the natural or processed state. Plant material generally includes leaves, fruit (both mature or ripe fruit, and immature or unripe fruit), stems, seeds, bark and roots, and the like that can be used in the preparation of the cranberry extract. The cranberry extract can be in the form of products including, for example, cranberry concentrates, cranberry fruits, cranberry fruit concentrates, cranberry juice, cranberry juice concentrate, cranberry juice extract, cranberry juice powder, cranberry powder, cranberry powder extract, and the like. Such products are commercially available from food product vendors such as, for example, Ocean Spray Cranberries, Inc. of Lakeville-Middleboro, Mass.

The cranberry extract can be in the form of a liquid, or as a powder that can dissolve to a large extent in a liquid within a reasonable period of time. The powdered cranberry extract can be processed in a form capable of being reconstituted as related to the mixing of dried fruit, vegetable and/or botanical powder with a liquid such as hot or cold water, milk or in some cases with semi liquid or other food ingredients to produce a reconstituted product. The reconstituted product can be a juice beverage, concentrate, sauce, pudding, soft drink, or a puree, and can be derived from natural, commercially available fruits, and/or vegetables and/or botanical plants.

There are known methods of partially or completely dehydrating such plant material and juices to produce solid form products. Typically plant material including fruit juices contain from about 84% to 92% of water. Conventional processes for producing dissolvable solid form products include, but are not limited to, spray drying, vacuum shelf or belt drying, drum drying, foam-mat drying, freeze drying and hydration drying. These processes generally involve the concentration of the juices followed by the dehydration of the concentrate through the application of heat and/or vacuum under controlled conditions.

The combination of trimethoprim and cranberry extract is effective for treating and preventing urinary tract infections. The combination can be formulated into pharmaceutical compositions. Standard pharmaceutical formulation techniques are used, such as those disclosed in Remington's Pharmaceutical Sciences, Mack Publishing Company, Easton, Pa., latest edition.

An “effective amount” of trimethoprim and cranberry extract is an amount, taken concurrently, that is effective to treat a urinary tract infections in warm-blooded animals, including humans, without undue adverse effects such as toxicity, irritation or allergic response, commensurate with a reasonable benefit/risk ratio when used in the manner of the present invention. The term “concurrently,” as used herein, means that cranberry extract and trimethoprim are administered within 24 hours of each other, preferably conjointly. The specific “effective amount” will, obviously, vary with such factors as the particular urinary tract infections being treated, the condition of the infection, the physical condition of the patient, the duration of treatment, the nature of concurrent therapy (if any), the specific dosage form to be used, the carrier employed, the solubility of dose form, potency of the cranberry extract, and the particular dosage regimen.

In addition to the effective amounts of the combination, the compositions of the present invention can further comprise a pharmaceutically acceptable carrier. The term “pharmaceutically-acceptable carrier,” as used herein, means one or more compatible solid or liquid filler diluents or encapsulating substances, which are suitable for administration to warm-blooded animals, including humans. The term “compatible,” as used herein, means that the components of the composition are capable of being commingled with trimethoprim and cranberry extract, and with each other, in a manner such that there is no interaction that would substantially reduce the pharmaceutical efficacy of the composition under ordinary use situations. Pharmaceutically acceptable carriers must, of course, be of sufficiently high purity and sufficiently low toxicity to render them suitable for administration to warm-blooded animals, including humans.

Some examples of substances which can serve as pharmaceutically-acceptable carriers or components thereof are: sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically acceptable carrier to be used in conjunction with the combination is basically determined by the way the composition is to be administered.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers. isotonic saline, and pyrogen-free water. Preferred carriers for parenteral administration include propylene glycol, ethyl oleate, pyrrolidone, ethanol, and sesame oil. Preferably, the pharmaceutically acceptable carrier, in compositions for parenteral administration, comprises at least about 90% by weight based on the total weight of the composition.

The compositions of the present invention can be in any of a variety of forms, suitable, for example, for oral administration. Depending upon the particular route of administration desired, a variety of pharmaceutically acceptable carriers well-known in the art can be used. These include solid or liquid fillers, diluents, hydrotropes, surface-active agents, and encapsulating substances. Optional pharmaceutically active materials can be included, which do not substantially interfere with the antimicrobial and antiadhesion activity of the combination. The amount of carrier employed in conjunction with the combination is sufficient to provide a practical quantity of material for administration per unit dose of the combination. Those of ordinary skill in the art know techniques and compositions for making dosage forms useful in the methods of the present invention.

The therapeutic compositions of the present invention are prepared simply by admixing trimethoprim and pharmaceutically acceptable salts thereof with the cranberry extract. The mixture is ultimately formulated with a pharmaceutically acceptable carrier admixed into a form suitable for administration to warm-blooded animals, including humans. The composition will generally be administered orally. Conventional methods such as administering the therapeutic actives in tablets, suspensions, solutions, emulsions, capsules, powders, syrups, and the like are suitable.

Various oral dosage forms can be used, including such solid forms as tablets, capsules, granules and bulk powders. Tablets can be compressed, tablet triturates, enteric-coated, sugar-coated, film-coated, or multiple-compressed. containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, and containing suitable solvents, preservatives, emulsifying agents, suspending agents, diluents, sweeteners, melting agents, coloring agents and flavoring agents.

In one embodiment of the present invention, the composition is an effervescent formulation comprising the trimethoprim and pharmaceutically acceptable salts thereof with the cranberry extract in combination with an effervescent agent. It has been proposed that dry effervescent formulations of trimethoprim and cranberry extract be prepared in which, upon addition to water, the cranberry extract and trimethoprim is dispersed in the water by the effervescing action and dissolves either as a result of the agitation or by interaction with components of the formulation.

The effervescent agent is preferably based on citric acid and sodium bicarbonate or sodium glycine carbonate, but other solid acid/carbonate couples can be used, for example tartaric or malic acid and sodium carbonate or potassium bicarbonate or mixtures of these acid and alkaline components. The effervescent agent is provided in a sufficient amount to rapidly disperse and assist dissolution of the components of the formulation. The corresponding potassium salts of the alkaline component can be used together with the sodium salts (or as a substitute) to avoid excessive levels of sodium ions.

Oral compositions in the form of liquid solutions, emulsions, suspensions, and the like are preferred. The pharmaceutically acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methylcellulose, sodium carboxymethyl cellulose, Avicel® RC-591, tragacanth and sodium alginate; typical wetting agents include lecithin and polysorbate 80; and typical preservatives include methyl paraben and sodium benzoate. Oral liquid compositions can also contain one or more components such as sweeteners, flavoring agents and colorants disclosed above.

The compositions of the present invention can be compressed by usual methods into solid oral dosage forms such as single or multilayer tablets. Moreover, the preparations can be produced in the form of coated tablets. Additionally, the preparations of the present invention can be provided in the form of hard shell capsules. It is also contemplated that the compositions of the present invention can be formulated into the form of dry powder mixes for preparing beverages containing the active ingredients. In general, the various oral dosage forms of the present compositions are prepared by conventional procedures and techniques of the art. The applicability of such methods and techniques to the formulation of the compositions of the present invention will be readily apparent to those skilled in the art.

The pharmaceutically acceptable carriers suitable for the preparation of unit dosage forms for oral administration are well known in the art. Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the present invention, and can be readily made by a person skilled in the art.

Such compositions can also be coated by conventional methods, typically with pH or time-pendent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit® coatings, waxes and shellac.

The pH of the compositions of the present invention can be adjusted by addition of a pharmaceutically acceptable acid or base. Suitable acids include, for example, hydrochloric acid and carboxylic acids such as citric acid, tartaric acid and succinic acid. Suitable bases include, for example, the oxides and hydroxides of calcium, potassium, sodium and magnesium, alkaline quanternary compounds, alkaline amino acids, and mixtures thereof. The compositions of the present invention are preferably pH balanced and/or buffered between about 5 to about 8.

It is also within the scope of this invention to administer each active component of the mixture individually. Thus, it is possible to formulate each of the components into separate dosage forms in accordance with procedures hereinbefore and hereinafter described for the combination.

Examples of sources of cranberry extract in a separate unit dose form include whole plant, including leaves (preferably), stems, shoots, berries, roots, and flowering parts, these can be ground, shredded or otherwise macerated and reduced in size for convenient use, and extracts thereof, as well as those unit dose forms commercially available including extracts, powders, capsules, gel caps, tablets, liquid, suspension, and tincture forms. Extracts can include both aqueous and organic solvent extract, e.g. ethanol, if desired, the extract can be dried and the resulting dried extract employed herein. For example, cranberry extract is available from Ocean Spray, Inc. Also encompassed are those commercially available products that contain at least some cranberry in part. Also further encompassed are capsules and tablets containing cranberry marketed for remedies for urinary tract infection.

The cranberry extract is incorporated in an amount of at least 5%, preferably ranging from about 10% to 95% by weight based on the total weight of the composition, and more preferably from about 25% to 50% by weight based on the total weight of the composition. Trimethoprim is typically present in the composition in an amount of from about 0.001% to 10% by weight based on the total weight of the composition, and preferably from about 0.01% to 5% by weight based on the total weight of the composition.

The ratios in which the trimethoprim to cranberry extract components are utilized in the preparation of the present invention can be varied within rather wide limits. Generally, the compositions of the present invention comprise typical ratio ranges of trimethoprim to cranberry extract ranging from about 1:2 to 1:100, preferably from about 1:5 to 1:40.

The compositions of the present invention can further be provided in a unit dosage form. As used herein, a “unit dosage form” is an amount of the combination that is suitable for administration concurrently to warm-blooded animals, including humans, in a single dose, according to good medical practice. These unit dosage forms preferably contain from about 1 mg to 10,0000 mg, more preferably from about 10 mg to 3,000 mg, more preferably from about 20 mg to about 2,000 mg of the combination. In an embodiment of a kit of the present invention where trimethoprim or pharmaceutically acceptable salts thereof, and cranberry extract are in separate unit dose forms, the two separate unit dose forms cumulatively contain the combination in the above-identified ranges.

The amount of trimethoprim in a unit dose will depend on the condition of the infection being treated. The unit-dose will be repeated according to the usual regime for trimethoprim treatments. Typically a unit dose can contain from about 50 mg to 400 mg of trimethoprim per dosage.

The compositions of the present invention can be achieved by incorporating unit dose forms of the combination into freeze-dried or lyophilized tablets or powder mixes. Freeze-drying or lyophilization facilitates disintegration of the composition by rapid permeation by the aqueous media, promoting timely delivery and dissolution of the product. Suitable methods of freeze-drying are well known in the art and commonly employed. Any suitable conventional method of freeze-drying can be utilized. A preferable method of freezing and drying is to fast freeze the composition and then dry the composition to a final moisture content of about 2% to about 5%.

Similarly, unit dose forms of the combination can be vacuum dried. Vacuum drying involves at least the partial drying of the compositions at temperatures above compositions' collapse temperature. Freeze drying, on the other hand, involves the drying of composition at temperatures below the composition's collapse temperature. Any suitable method of vacuum drying can be used.

In another embodiment of the present invention, there is provided a method for treating or preventing a urinary tract infections in warm-blooded animals, including humans, comprising administrating a therapeutic composition comprising a combination of trimethoprim or pharmaceutically acceptable salts thereof and cranberry extract, and a pharmaceutically acceptable carrier, to the warm-blooded animal in an effective amount for a time sufficient to prevent, reduce or eliminate symptoms associated with the infection.

The treatment in accordance with the present invention renders bacteria non-pathogenic and unable to colonize the urinary tract. Thus, one measure of efficacy includes monitoring the reduction or elimination of urinary bacterial counts associated with such infections during or after the course of treatment.

The compositions of the present invention can be administered to treat and prevent urinary tract infections in a subject in need thereof. As used herein, a “urinary tract infection” is any disorder characterized by the presence of a microbial infection of the urinary tract. Included within bacterial infections are those infections caused by Pseudomonas aeruginosa, Serratia marcescens, Enterococcus Faecalis, Klebsiella pneumoniae, Proteus mirabilis, Escherichia coli, Staphylococcus saprophyticus and others known to be implicated in UTIs.

One skilled in the art would readily identify a urinary tract infection. For example, the diagnostic techniques for a UTI include, but are not limited to, palpation over the kidney, urinalysis, urine culture (clean catch), urine culture (catheterized specimen), blood culture, intravenous pyelogram scan, computed tomography scan, voiding cystourethrogram, renal ultrasound, renal scan, and renal biopsy.

Symptoms of urinary tract infections are readily identifiable by those skilled in the art, and also by lay consumers. For example, the symptoms of UTI include, but are not limited to, pressure in the lower pelvis, painful urination (dysuria), frequent need to urinate, urgent need to urinate, need to urinate at night, cloudy urine, blood in the urine (hematuria), and foul or strong urine odor. Fever may or may not be a feature at presentation.

The term “treatment” is used herein to mean that, at a minimum, administration of a composition of the present invention mitigates urinary tract infections in warm-blooded animals, including humans. Thus, the term “treatment” includes: preventing a urinary tract infections from occurring in a warm-blooded animal, particularly when the warm-blooded animal is predisposed to acquiring the urinary tract infection, but has not yet been diagnosed with the disease, inhibiting the urinary tract infection; and/or alleviating or reversing the urinary tract infection.

Insofar as the methods of the present invention are directed to preventing a urinary tract infection, it is understood that the term “prevent” does not require that the urinary tract infections be completely thwarted. (See Webster's Ninth Collegiate Dictionary.) Rather, as used herein, the term “preventing” refers to the ability of the skilled artisan to identify a population who is susceptible to urinary tract infections, such that administration of the compositions of the present invention can occur prior to the onset of the symptoms of the urinary tract infections. The population that is at risk for a urinary tract infection, include those who are subjected to: an obstruction of the bladder or urethra with resultant stasis of urine, insertion of instruments into the urinary tract (such as catheterization or cystoscopy), pregnancy, diabetes, and a history of analgesic nephropathy or reflux nephropathy. The elderly population is at increased risk for developing an UTI due to lack of mobility and/or incomplete emptying of the bladder associated with such conditions are benign prostate hyperplasia, prostatitis, and urethral strictures.

Thus, the patient population is identifiable and could receive the administration of a composition of the present invention before progression of the disease. Thus, progression of the urinary tract infections in such individuals would be “prevented.”

The compositions of the present invention are also useful for prophylactic or acute treatment. The compositions of the present invention are administered in any way the skilled artisan in the field of medicine or pharmacology would desire. It is immediately apparent to the skilled artisan that preferred routes of administration depend on the stage of the urinary tract infections being treated and the dosage form chosen. A preferred route for systemic administration is an oral route.

The compositions of the present invention can be administered systemically. Systemic application includes any method of introducing a composition of the present invention into the tissues of the body, for example, epidural, intramuscular, transdermal, intravenous, intraperitoneal, subcutaneous, sublingual, rectal, and oral administration. The compositions and unit dose form of kits of the present invention are preferably administered orally.

The specific dose of a composition of the present invention to be administered, as well as the duration of treatment, is mutually dependent. The dosage and treatment regimen will also depend upon such factors as the route of administration, the type of dosage form used, the infectious agent present, the ability of the combination to reach and sustain effective levels at the site of infection, the nature and extent of other infections (if any), the personal attributes of the subject (such as weight), compliance with the treatment regimen, and the presence and severity of any side effects of the treatment.

For systemic administration of the compositions, typically for a human adult (weighing approximately 70 kilograms), from about 1 mg to about 10,000 mg, preferably from about 10 mg to about 5000 mg of the compositions are administered per day.

The foregoing notwithstanding, it should be fully understood that the dosages set forth herein are exemplary only and they do not, to any extent, limit the scope of practice of the present invention.

Treatment regimens preferably extend from about once to about five times daily, for about 1 to about 56 days, preferably for about 3 to about 10 days. It is understood that these dosage ranges are by way of example only, and that daily administration can be adjusted depending on the factors listed above. It is believed that cranberries and extracts thereof promote anti-adhesion of microbial pathogens in warm-blooded animals, including humans and particularly in the urinary tract. Accordingly, it is highly recommended that a plentiful amount of fluids preferably water be administered during the course of the treatment regimen. This intake of fluids promotes the cleansing and flushing of the urinary tract. The amount of fluid intake can be from at least 1.0 L per day, preferably from about 1.5 L to 3.5 L per day, and more preferably from about 1.8 L to 2.3 L per day, depending on several factors including the weight of the patient, the physical activity of the patient, the ambient temperature, and the like.

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

What is claimed is:

1. A method for making an over-the-counter therapeutic composition useful for treating or preventing urinary tract infections in warm-blooded animals, including humans, comprising the steps of:
obtaining a combination of trimethoprim or pharmaceutically acceptable salts thereof in amounts ranging from about 50 mg to 100 mg, an effective amount of cranberry extract, and a pharmaceutically acceptable carrier to yield a dry mixture; and
mixing at least one liter of liquid with the dry mixture to yield the therapeutic composition.

2. The method of claim 1, wherein the liquid is water.

3. The method of claim 1, further comprising adding an effervescent agent to the composition.

4. The method of claim 1, wherein the ratio of trimethoprim and cranberry extract is from about 1:2 to 1:100 by weight based on the total weight of the composition.

5. The method of claim 1, wherein the ratio of trimethoprim and cranberry extract is from about 1:5 to 1:40 by weight based on the total weight of the composition.

6. The method of claim 1, wherein the liquid is present in the amount of from about 1.5 L to 3.5 L.

7. The method of claim 1, wherein the liquid is present in the amount of from about 1.8 L to 2.3 L.
